(19) United States
(12) Patent Application Publication
(10) Pub. No.: US 2004/0111048 A1
(43) Pub. Date: Jun. 10, 2004

(54) Compression device for treatment of chronic venous insufficiency

(76) Inventors: Jeffrey L. Jensen, Evergreen, CO (US); Paul P. Burek, Centennial, CO (US); Daniel J. Macfarlane, Littleton, CO (US)

Correspondence Address: Gibson, Dunn & Crutcher LLP, Suite 4100, 1801 California Street, Denver, CO, 80202, US

(21) Appl. No.: 10/309,483

(22) Filed: Dec. 4, 2002

(51) Int. Cl. A61F 5/00

(52) U.S. Cl. 602/13

(57) ABSTRACT

The present invention provides a medical device and treatment method for chronic venous insufficiency and related medical conditions. The device is an inflatable stocking which, when inflated, applies pressure to the foot and lower leg of a patient. The device has several sections that are inflatable to different pressures so that gradient pressure may be applied. The sections are vertically disposed so that greatest pressure is applied to the foot and lower portion of the lower leg, somewhat less pressure is applied to the middle portion of the lower leg, and the least pressure is applied to the upper section of the lower leg. The device surrounds the entire treatment area so that the treated area will not swell.

### FIELD OF THE INVENTION

The present invention relates to medical devices and treatments for chronic venous insufficiency and related medical conditions, and more particularly to a device and treatment incorporating an inflatable compression device capable of applying gradient compression to the foot and lower leg of a patient.

### BACKGROUND

Chronic venous insufficiency (CVI) is a significant and growing medical problem. The pathophysicologic basis of CVI is venous hypertension in the lower extremities. The calf-muscle pump works by contracting around veins in order to force blood in the veins into motion. One-way valves within the deep venous systems allow blood to flow only proximally out of the legs. Failure of these valves leads to increased venous hypertension in the superficial system, thereby decreasing calf-muscle pump efficiency. Increasing venous distension can promote increasing valvular incompetence, leading to symptoms such as leg swelling and aching, discoloration of skin, activity intolerance, and finally open ulceration. Increased venous pressure results in extravasation of fluid, serum proteins, and blood cells into the subcutaneous tissue, eventually leading to pigmentation changes and ulceration. The high prevalence and resulting costs of venous pathology, such as health care costs, missed work, and reduced quality of life constitute a heavy burden on society. Approximately 5 million Americans exhibit some evidence of CVI and, depending on estimates, between 500,00 and 600,000 or up to one million of these individuals have or will develop venous leg ulcers, causing recurrent hospitalization, high health care costs, and disability. Fifty percent of venous ulcers may be present for 7-9 months. Between 8 and 34% of the ulcers may be present for more than 5 years, and 67-75% of patients have recurrent problems. An estimated two million workdays are lost each year in the United States. The medical costs of treatment and indirect costs associated with the disease can be significant. According to a study performed at the Cleveland clinic, the medical cost per venous leg ulcer averaged nearly $10,000.

While the etiology and pathophysiology of CVI and resulting venous ulcers are well established, there has not been satisfactory progress in the treatment of this problem. It is in response to CVI and resulting venous ulcers that the present invention arises.

It is known to be beneficial to use compression of the foot and lower leg in the treatment of CVI. It is believed that the application of external pressure to the calf muscle raises the interstitial pressure, forcing blood in the deep venous system, decreasing the superficial venous pressure and improving venous return that leads to a reduction in superficial venous hypertension. This allows ulcers to heal. Gradient compression has been achieved using a “Jobst stocking”, i.e., a compressive garment (related to compression bandages and hosiery) that is worn around the foot and lower leg. Compression techniques have long been used in a number of different treatment regimes, with a reasonable degree of success when there is good patient compliance. Unfortunately, compression has not proven efficacious in poorly compliant patients, who universally have a high rate of ulcer persistence or recurrence. Several factors contribute to poor patient compliance. Often patients do not have enough strength or mobility to pull on compression stockings. Attempts have been made to overcome these difficulties, such as by the u-se of a zippered back (Jobst), or leggings with a series of interlocking bands fastened with hoop and loop fasteners (CircAid). However, even these improvements have not been successful in solving the problem of poor patient compliance.

Such compression garments may be ineffective in patients with massive edema or obesity, as the garments lose their elasticity over time. By the end of the day, edema often returns along with symptoms. As a result of a loss in elasticity, these garments must be replaced frequently—generally every three or four months.

Inflatable garments have also been used to apply compression to the foot and lower leg in a non-ambulatory setting. However, such devices do not provide gradient compression, which limits their effectiveness. Finally, sequential compression pumps are used to “milk” fluid in the legs proximally. However, such pumps are only effective while worn by the patient, and are not a viable long-term treatment option, as they do not allow ambulation while being worn.

It is clear that a device capable of applying gradient compression in an ambulatory patient while overcoming the shortcomings of known compression devices will be a welcome advance in the treatment of CVI.

### SUMMARY

The present invention provides a device which can be worn like a sock, and which uses air (or other fluid) pressure to apply gradient compression to the foot and lower leg of a patient suffering from CVI or a similar ailment. The device allows for ambulation of the patient, who may use the device with normal shoes. The device is sufficiently comfortable to be worn for an entire day, and is durable and washable. (The device may be washable in its entirety if any electronic components are immersible, or electronic components may be removed before washing.) The device is easy to put on correctly and remove even by those with physical infirmities. This ease in application overcomes a significant problem faced by prior compression devices and is expected to provide much higher patient compliance than was previously possible.

The device has an inflatable bladder that fits over the upper portion of the foot and lower leg of the patient. A flexible non-elastic outer sleeve surrounds the bladder, so that inflation of the bladder compresses the foot and lower leg of the patient. The outer sleeve can be a pliable, semi-soft “shell.” The bladder surrounds the foot and leg with no unenclosed areas in the treatment portion of the foot and leg, and the device is securely closed with hook and loop fasteners to avoid swelling of other areas of the foot and/or leg which could otherwise result. The bladder does not extend to the bottom of the foot, which allows normal loose fitting shoes to be worn. The bladder partially covers the top of the foot.

The bladder includes three sections (a bottom section, middle section, and top section) that are inflated to separate pressures to provide gradient compression, such as 30-40 mmHg at the foot and ankle, 20-30 mmHg at the mid-lower leg, and 10-20 mmHg at the upper-lower leg. In an embodiment, the sections are connected via pressure differential valves.

To use the device, the patient first places it around his foot and lower leg. The patient then closes the device using the foot hook and loop closure. Next, the patient inflates the bladder via an air pump connected to an inlet valve connected to the bottom section. As the bottom section is inflated to its target pressure, the pressure differential valve connecting the bottom and middle section opens. The middle section then fills to its target pressure. A pressure differential valve between the second and third section allows the upper section to fill to its target pressure. A pressure relief valve on the top section ensures that the pressures never exceed predetermined safety levels.

An air pump and pressure switch are positioned either at the top of the device, or positioned elsewhere such as on the user's waist. The pump inflates the bladders and, in a preferred embodiment, maintains the desired pressure over time.

Following a full day of wear, the user simply deflates the bladder, unfastens the foot hook and loop closure, leaving the upper closures in place, and removes the device. This is easily accomplished, even by the infirmed. The device is designed so that it can be applied and removed without the assistance of adjunct personnel. It should be understood that not all embodiments are described in this summary.

### BRIEF DESCRIPTION OF THE DRAWINGS

FIG. 1 is a perspective view of an embodiment of the invention.

FIG. 2 is a perspective view of an embodiment of the invention, showing in particular the bladder system.

FIG. 3 is a cross section of an embodiment of the invention, taken at a position corresponding to a user's mid-calf.

FIG. 4 is a cross section of an embodiment of the invention, taken at a position corresponding to a user's ankle.

FIG. 5 shows a user wearing an embodiment of the invention.

FIG. 6 is a perspective view of another embodiment of the invention, showing in particular the bladder system.

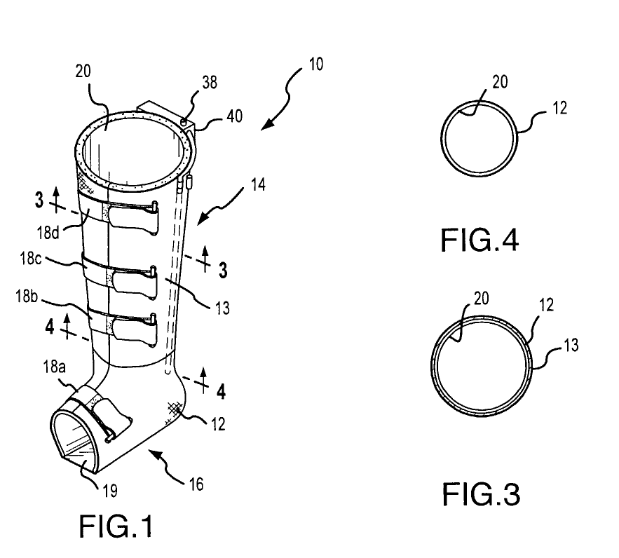

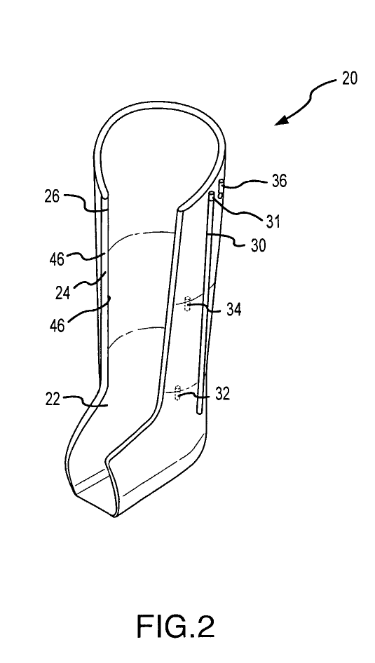

### DETAILED DESCRIPTION

The reference characters designate the following:

10 device

12 outer sleeve

13 supporting layer

14 upper section

16 lower section

18 closure system

18a-d closure sections

19 section of outer sleeve under foot

20 bladder system

22 lower bladder section

24 middle bladder section

26 upper bladder section

30 tube

31 air inlet valve

32, 34, pressure differential valves

36 relief valve

38 pump air inlet

40 pump pressure switch

42 bladder outer wall

44 bladder system inner wall

46 material defining bladder sections

P pump

The present invention is a device and method of using the device to treat and prevent CVI and related ailments. In overview, the device includes a bladder which when inflated applies compressive force to the foot and lower leg of the patient. Separate bladder sections allow for gradient compression, which is more beneficial than applying a single pressure to the treatment area.

The device 10 includes a flexible, inelastic outer sleeve 12 that encloses the foot and lower leg of the patient. The outer sleeve supports and contains an inflatable bladder system 20, which transfers compressive forces to the foot and lower leg.

The outer sleeve 12 is preferably constructed of lightweight, inelastic structural fabric that is durable and weather resistant. Examples of such fabrics are polyester, nylon, and GORE-TEX (TM). The outer sleeve 12 is generally conically shaped to match the contours of the human leg, so that it can be placed over the leg and lower foot in the manner of a stocking. As can be seen, the sleeve has an upper section 14, which fits over the lower leg, and a lower section 16, which fits over the foot. The lower section 16 extends in a general “L” bend from the upper section 14. The lower section 16 extends for a distance to cover a major portion of the top of the foot, but not the toes.

Referring to FIG. 3, in a preferred embodiment, a supporting layer 13 of thin foam surrounds the outer sleeve 12 to provide additional rigid support. Materials other than thin foam that provide rigidity could also serve this purpose. Preferably, the supporting layer 13 does not surround the entirety of the outer sleeve 12, but instead only surrounds the portion of the upper section 14 above the user's ankle. See also FIG. 4, showing a cross section taken at the user's ankle. Without the supporting layer 14, the device 10 may slide down a user's leg, at least in some cases. The foam thickness may be about 1/16″ thick, but could be thicker such as about ⅛″ thick, depending upon its characteristics and the specific embodiment of the invention. The important point is that the foam is thick enough to keep the device 10 from “falling down”, yet thin enough to be pliable enough to conform to the user's leg.

A vertically oriented hook and loop closure system 18 allows the outer sleeve 12 to be wrapped around the leg and foot, and then closed by fastening the hook and loop system 18S. As shown, the hook and loop closure system 18 is composed of four vertically oriented sections, 18a, 18b, 18c, and 18d, although it will be clear that a single continuous length of hook and loop closure could be used or some other number of closures could be used. It will also be clear that other methods of closing fabric, such as zippers, straps, buckles, or other closure means could be used. However, hook and loop closures are probably the easiest for a patient to use and allow for quickly and securely applying and removing the device. The hook and loop closure sections 18a, 18b, 18c and 18d are sewn or otherwise attached to the remainder of the outer sleeve 12.

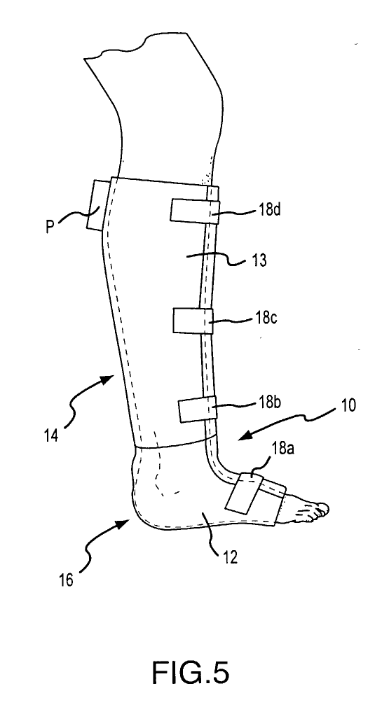

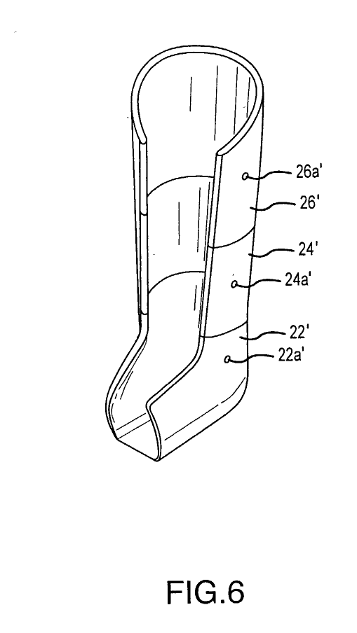

In use, a clinician preferably fits the top there sections 18b, 18c, and 18d when the device 10 is fitted on a particular patient. Once fit, they are not further adjusted by the patient (of course, if they are causing discomfort or the fit is not optimal, they can be refit). The bottom (foot) closure 18a is unfastened by the user to take the device 10 off and on, and is fastened in normal use. FIG. 5 shows the device 10 as fit onto the leg and foot of a typical patient.

When the user wants to wear a shoe while wearing the device 10, most preferably the user first puts on the device, then the shoe, then inflates the device. Inflation is discussed below.

The outer sleeve 12 has a flat and thin section 19 that fits underneath the foot, allowing the user to wear a shoe over when the device 10 is worn.

The bladder system 20 is the primary component ensuring efficacy of the device. The bladder system 20 is shaped to contact the foot except the forefoot, the bottom of the foot, and a section of the sides of the foot. The bladder system 20 is shaped to exert pressure around the entire lower leg and foot proximal to the 1st metatarsal head medially and the 5th metatarsal head laterally. As with the sleeve, the bladder system 20 is circumferential (when the device is worn by the user) so that pressure is applied around the entire portion of the leg covered by the bladder.

The bladder has three separate compartments: a lower section 22, a middle section 24, and an upper section 26. An air inlet valve 31 is connected to the lower section 22 via a tube 30, discussed in more detail below. The lower section 22 is connected to the middle section 24 via a pressure differential valve 32, and the middle section 24 is connected to the upper section by a pressure differential valve 34. A pressure relief valve 36 is connected to the upper section 24. The characteristics of the valves 32, 34, and 36 can be chosen to allow for any pressures to be maintained in the three bladder sections 22, 24, and 26. In a presently preferred design, the valves 32, 34, and 36 are selected so that, when inflated, the pressure in the lower section 22 will be between 30-40 mmHg, the pressure in the middle section 24 will be between 20-30 mmHg, and the pressure in the upper section 26 will be between 10 and 20 mmHg. Vernay Laboratories, Inc. manufactures valves that are suitable for the device 10, although such valves are also available from other sources.

In operation, the three-chamber bladder system allows these pressures to be applied and maintained. As the lower chamber 22 is inflated (by pumping air through the inlet valve 31, as described in more detail below) the valve 32 is opened to let air in the middle bladder. As air enters the middle bladder 24, the pressure increase forces the valve 34 to open, thereby letting air in the top bladder 26. All three bladders are filled until the top bladder reaches 10-20 mmHg pressure. This is reached when the relief valve at the top bladder is opened, which indicates that there is 10-20 mmHg pressure. The valve 34 closes when there is a 10 mmHg pressure differential between top and middle bladders. This point is when the middle bladder is at 20-30 mmHg pressure. The valve 32 closes when the pressure in the lower bladder is 30-40 mmHg (10 mm pressure differential). As described in more detail below, a pump can be automatic to maintain the pressure in the lower bladder to 30-40 mmHg against the lower leg and foot.

The thickness of the bladder system 20 can be as desired; in a preferred design it is about 1/4 inch thick, which allows the patient to walk comfortably. The lower section 22 is shaped to thin out toward its distal end (see 19) which, when applied to the patient, approaches the plantar portion of the foot. Thus a user can wear the device 10 underneath a conventional, loose fitting shoe.

The bladder system 20 is preferably made of two types of material welded together by RF welding or otherwise attached to form a closed pneumatic system. The bladder system outer wall 42 is polyurethane film or similar material. The bladder system inner wall 44 may be made of a stretchable material such as Lycra and polyurethane composite, which aids in conforming the bladder system to the contours of the foot and leg, or a polyurethane coated polyester felt, which may be inelastic. RF welding can be used to form the three sections 22, 24, 26 such as by welding a ⅛″ section of material 46 between the lower section 22 and the middle section 24, and between the middle section 24 and the upper section 26. This technique is known in the manufacture of air bladders used in athletic shoes and fracture casts. The air bladder system 20 is attached to the outer sleeve via adhesives or by any other suitable attachment means.

Any pump system may be used to inflate the air bladder system. As only air pressure is required, a small, lightweight, quiet, and inexpensive air pump is all that is required. In a preferred embodiment, a pump P is attached to the upper back of the device 10. The pump P has an air inlet 38 for drawing air from the atmosphere. The pump P is engaged (via appropriate tubing or other connection) with a tube 30. The tube 30 connects the pump P to the lower bladder section 22. The top of the tube 30 houses an air inlet valve 31, allowing for inflation of the lower bladder section 22 and, because of the interconnections, the entire bladder system 20.

The tube 30 is preferably located between the bladder system 20 and the outer sleeve 12 to avoid direct contact with the user and so that the outer sleeve 12 protects the tube 30, but this is not essential. The pump P includes a pressures switch 40 in communication with the air inlet valve 31. The switch 40 switches the pump on when the pressure in the lower bladder section 22 is below a threshold (e.g., 30 mmHg) corresponding to the lower bladder section desired range. The switch 40 is generally off otherwise (it can operate like a thermostat so that it is not frequently cycling on and off; for example, it could inflate to 40 mmHg when it is on, but only turn on when pressure drops below 30 mmHg). The pump P maintains the bladder system 20 at the desired pressures. For instance, if the lower bladder 22 loses air pressure (such as bleeding air into the middle chamber 24), the pressure switch 40 senses this pressure drop and the pump turns on to add air into the lower chamber 22. Similarly, a reduction in pressure in the middle bladder 24 will self correct because the valve 32 will cause air to be added from the lower bladder 22. And a reduction in pressure in the upper bladder 26 will self correct because the valve 34 will cause air to be added from the middle bladder 24. Because the pump adds air to the lower bladder 22 when necessary, all three bladders 22, 24, and 26 will automatically stay within the desired pressure range. The user may deflate the bladder system 20 by opening the relief valve 36.

As shown, the pump P is attached to the device 10, but it could also be elsewhere, such as on the user's waist. Appropriate tubing connects the pump P to the air inlet valve 31. In another embodiment, the user attaches the pump P to the air inlet valve 31 whenever inflation is desired, such as when the user first applies the device 10 (preferably after first putting on a shoe, if a shoe is to be worn). In this embodiment, the user may or may not reattach the pump P to the air inlet valve 31, as desired. Either a powered pump or a hand pump (such as bulb-type hand pump) could also be used. The pump may have a gauge so the patient will know when to stop inflating the bladder system. Alternately, the patient can fill until the relief valve 36 starts bleeding air.

In use, a patient having venous stasis ulcers or other conditions requiring a dressing may use the device. The primary dressing (i.e., dressing immediately contacting the wound) can by any of a number of wound-healing modalities including, but not limited to, amorphous hydrogel, calcium alginate, polyurethane foam, growth factor, and synthetic skin equivalents. This may be followed by a Kerlix wrap or equivalent. Thus, the device does not contact an open wound, thus avoiding biocompatibility issues of the wound or wound-healing compounds.

The device can be used in a number of treatment modalities but it is contemplated that the device will be applied by the patient and inflated at the start of the day, worn all day, and deflated and removed when the patient goes to bed.

Another embodiment of a bladder system is shown in FIG. 6. In that embodiment, the lower section 22′, middle section 24′ and upper section 26′ each have a separate inlet 22a′, 24a′, and 26a′ respectively. The sections 22′, 24′, and 26′ are not in communication with each other. Instead, each chamber is inflated separately to the desired pressures mentioned above, or to any other desired pressure. In this manner, gradient pressure can be applied in a device that is somewhat simpler to manufacture, but somewhat less convenient for the user. In operation, the user attaches a pump to each of the inlets 22a′, 24a′ and 26a′, and inflates them separately.

The efficacy of an embodiment of the device has been demonstrated by the results shown in the following table.

TABLE
Average Gradient Pressures at Application

| | | Gradient | | Gradient | |
|---|---|---|---|---|---|
| | FOOT/ANKLE | Change | MID-CALF | Change | UPPER CALF |
| DESIRED | 30-40 mmHg | | 20-30 mmHg | | 10-20 mmHg |
| PRESSURE | | | | | |
| JOBST | 35.12 mmHg | 11.68 mm | 23.44 mmHg | 3.03 mm | 20.41 mmHg |
| CIRCAID | 27.61 mmHg | 0.34 mm | 27.27 mmHg | 2.84 mm | 24.43 mmHg |
| SOC | 38.61 mmHg | 11.05 mm | 27.56 mmHg | 9.19 mm | 18.37 mmHg |

The device 10 applies meaningful gradient pressure over a period of time. The device tested in FIG. 7 did not have an automatic pump, which would have solved the problem of decreasing pressure over time.

What is claimed is:

1. A device for applying gradient pressure to the foot and lower leg of a patient, comprising: an inflatable bladder that is generally circumferentially shaped so that it can be fit over at least a portion of the foot and lower leg of a patient, the inflatable bladder having at least an upper section and a lower section that are inflatable to different pressures so that when so inflated gradient pressure is applied.

2. The device of claim 1 further comprising a middle section between the upper section and the lower section that is inflatable to a pressure different from the upper section and the lower section.

3. The device of claim 2, wherein the bladder sections are connected by pressure differential valves.

4. The device of claim 2, wherein one bladder section has an air inlet valve and another bladder section has a pressure relief valve.

5. The device of claim 4, wherein the lower section has the air inlet valve and the upper section has the pressure relief valve.

6. The device of claim 3, wherein the bladder is surrounded by an outer shell that is sufficiently inelastic so as to maintain constant pressure applied by each section.

7. The device of claim 6, wherein the bladder is flexible to conform to the foot and leg of the patient.

8. The device of claim 3, wherein the lower section is inflatable to 30-40 mmHg, the middle section is inflatable to 20-30 mmHg, and the upper section is inflatable to 10-20 mmHg.

9. The device of claim 8 wherein one section has a pressure relief valve and the combination of the pressure relief valve and the pressure differential valve cause pressure to be released from any section having a pressure that would otherwise be above the specified parameters.

10. The device of claim 3 further comprising a hook and loop closure.

11. The device of claim 3, further comprising a supporting layer surrounding the outer sleeve to provide additional support so that the device will not slide down the foot and lower leg of the patient.

12. The device of claim 11, wherein the supporting layer is a layer of relatively thin foam.

13. The device of claim 3, further comprising a pump having a pressure switch in gaseous communication with inflatable bladder so that air will be pumped into the bladder when the switch senses a pressure below a pre-defined threshold.

14. The device of claim 13, wherein the pump pressure switch is in gaseous communication with the lower chamber.

15. A method for applying gradient pressure to the foot and lower leg of a patient, comprising: applying to the patient an inflatable bladder that is generally circumferentially shaped so that it can be fit over at least a portion of the foot and lower leg of a patient;
inflating a lower bladder section to a first pressure;
inflating an upper bladder section to a second pressure that is lower than the first pressure.

16. The method of claim 15, comprising the step of inflating a middle bladder section that is located between the lower bladder section and the upper bladder section to a third pressure that is between the first and second pressures.

17. The method of claim 16, wherein the upper, lower, and middle sections are connected by pressure differential valves and the inflating step involves applying a pump to one of the sections.

18. The method of claim 17, wherein the pump is applied to the lower section.

19. The method of claim 17, wherein the upper section has a pressure relief valve to prevent over inflation, and wherein the lower section is inflated to 30-40 mmHg, the middle section is inflated to 20-30 mmHg, and the upper section is inflated to 10-20 mmHg.

20. The method of claim 17, further comprising connecting a pump having a pressure switch to the inflatable bladder for pumping air into the bladder when the switch senses a pressure below a pre-defined threshold.

21. The method of claim 17, wherein an outer shell surrounds the bladder.

22. The method of claim 21, wherein the outer shell is closed using a hook and loop closure.

23. The method of claim 21, wherein a supporting layer surrounds the outer layer.
